(19) United States
(12) Patent Application Publication
(10) Pub. No.: US 2024/0299765 A1
(43) Pub. Date: Sep. 12, 2024

(54) LIGHT THERAPY DEVICE

(71) Applicant: Mito Red Light, Inc., Scottsdale, AZ (US)

(72) Inventor: Scott Chaverri, Scottsdale, AZ (US)

(21) Appl. No.: 18/578,304

(22) PCT Filed: Nov. 21, 2022
(86) PCT No.: PCT/US2022/050625

§ 371 Date: Jan. 10, 2024

Related U.S. Application Data
(60) Provisional application No. 63/337,165, filed on May 1, 2022.

(51) Int. Cl. A61N 5/06; A61N 5/00

(52) U.S. Cl. CPC A61N 5/06; A61N 2005/005; A61N 2005/0626; A61N 2005/0652; A61N 2005/0659; A61N 2005/0663

(57) ABSTRACT

A light therapy device and methods of use for delivering photobiomodulation “light therapy” is provided. The light therapy device includes hardware and functionality to deliver a wide variety of therapeutic programs via operating modes, wherein specific combinations of LED chips are powered to generate customized ranges of light spectra to the user. Example embodiments for “stand alone” use as well as embodiments for mounting in a building structure or furniture, for fashioning into garments and the like are disclosed.

### CROSS REFERENCE TO RELATED APPLICATIONS

This application claims the benefit of U.S. provisional application No. 63/337,165 entitled “Light Therapy Device.” filed on May 1, 2022, the enclosures of which are incorporated entirely by reference herein.

### BACKGROUND OF THE INVENTION

### Technical Field

The disclosures herein relate to light therapy devices. Specifically, the disclosed invention relates to a device and methods for delivering phototherapy. The devices and methods incorporate various wavelengths, frequencies, luminosities, and combinations thereof for supporting or improving a variety of health states and medical conditions.

### BRIEF SUMMARY

Disclosed herein are embodiments of a device and methods for delivering light therapy.

Low-level laser light therapy (“LLLT”) was discovered in 1967 by Endre Mester at the Semmelweis Medical University in Hungary. Mester was trying to repeat an experiment using high powered lasers to cure tumors in rats. Despite failing to cure tumors with his low-powered laser, Mester observed improved hair growth and enhanced wound healing in the rats. This was the first indication that low-level laser light, rather than high power thermal lasers, could have beneficial applications.

It is now widely understood that devices utilizing light-emitting diodes (“LEDs”) to illuminate living tissue can be used to obtain beneficial biological effects. Accordingly, the term “LLLT” has been gradually replaced in the literature with the broader term “photobiomodulation” (“PBM”). As the health benefits of PBM have become more widely known, there has been a proliferation of both commercial and consumer PBM LED devices in the marketplace.

Due to limitations inherent in LED chip technology, however, existing devices are markedly limited in both the range of light wavelengths and functionality. Because skin and subcutaneous tissues manifest different absorption characteristics for light comprising wavelengths within the optical window, the literature has repeatedly shown that different wavelengths may be more (or less) applicable for different purposes. Additionally, the human brain, along with the nervous, and endocrine systems, responds in complex ways when exposed to varying light spectra throughout the 24-hour circadian wake-sleep cycle. Luminosity, time duration of exposure, duty cycle duration and interval times, combinations of wavelengths, delivery modalities, and other variations in how PBM therapies are delivered are also important.

Existing PBM devices, although able to deliver light therapy comprising limited combinations of wavelengths and pulse durations, lack the functionality to provide an expansive combination of wavelength spectra and pulse durations. Consequently, there is a need for PBM therapy delivery systems and devices that can deliver highly specific PBM therapies necessary to address the specific health needs of an individual user.

A light therapy device having functionality to deliver electromagnetic radiation in the infrared and near-infrared spectrum is disclosed herein.

Disclosed is a light therapy device comprising a case having an interior defined by at least one side and configured to mount a plurality of light emitting diode (LED) assemblies within the interior, wherein each LED assembly comprises a light source mounted on a printed circuit board, and a reflector; and at least one operating mode, wherein the at least one operating mode comprises emission of at least one spectra of electromagnetic radiation from a plurality of LED assemblies comprising a peak spectral wavelength between about six hundred nanometers (600 nm) and about one thousand nanometers (1,000 nm).

In some embodiments, fifty percent (50%) of the plurality of LED assemblies comprise a light source having a peak intensity wavelength of about 630 nm. In some embodiments, fifty percent (50%) of the plurality of LED assemblies comprise a light source having a peak intensity wavelength of about 660 nm. In some embodiments, fifty percent (50%) of the plurality of LED assemblies comprise a light source having a peak intensity wavelength of about 810 nm. In some embodiments, fifty percent (50%) of the plurality of LED assemblies comprise a light source having a peak intensity wavelength of about 850 nm. In some embodiments, fifty percent (50%) of the plurality of LED assemblies comprise a light source having a peak intensity wavelength of about 940 nm.

In some embodiments, the light therapy device further comprises a computing module having at least one software algorithm residing on a memory, wherein the at least one software algorithm causes the light therapy device to execute at least one operating mode.

In some embodiments, the at least one operating mode is executed in response to instructions received from a wireless remote handheld device having a user interface.

In some embodiments, the at least one operating mode comprises simultaneously powering equal numbers of the plurality of LED assemblies having a peak wavelength intensity of about 630 nm, the plurality of LED assemblies having a peak wavelength intensity of about 660 nm, the plurality of LED assemblies having a peak wavelength intensity of about 810 nm, and the plurality of LED assemblies having a peak wavelength intensity of about 850 nm.

In some embodiments, the at least one operating mode comprises simultaneously powering a plurality of LED assemblies having a peak wavelength intensity of about 630 nm, a plurality of LED assemblies having a peak wavelength intensity of about 810 nm, and a plurality of LED assemblies having a peak wavelength intensity of about 850 nm at a ratio of 2:1:1.

In some embodiments, the at least one operating mode comprises comprises simultaneously powering a plurality of LED assemblies having a peak wavelength intensity of about 630 nm, a plurality of LED assemblies having a peak wavelength intensity of about 660 nm, and a plurality of LED assemblies having a peak wavelength intensity of about 850 nm at a ratio of 1:1:2.

Disclosed is a light therapy device comprising a module having a plurality of light emitting diode (LED) assemblies arranged in a pattern forming an array, wherein the plurality of LED assemblies are mounted on a printed circuit board (PCB), a heat sink, a cooling fan, and a transformer electrically coupled to the PCB, wherein the transformer is configured to power the plurality of LED assemblies; and a case having an interior defined by at least one side and configured to mount a plurality of modules.

The foregoing and other features and advantages of the invention will be apparent to those of ordinary skill in the art from the following more particular description of the invention and the accompanying drawing figures.

### BRIEF DESCRIPTION OF THE DRAWINGS

FIG. 1A is a perspective view of an example embodiment of a light therapy device;

FIG. 1B is a perspective view of an example of multiple light therapy devices electrically and communicatively coupled together;

FIG. 2 is a perspective-view diagram of an LED assembly used in some embodiments of a light therapy device;

FIGS. 3A-B are examples of LED arrays used in some embodiments of a light therapy device;

FIG. 4 is a diagram of a module used in some embodiments of a light therapy device;

FIG. 5 is a diagram of a computing module used in some embodiments of a light therapy device;

FIGS. 6-16 are charts of different examples of LED assembly activation wavelengths and combinations showing some example embodiments of operating modes of a light therapy device; and

FIG. 17 is a graph of irradiance plotted against wavelength for example spectra utilized in six (6) separate operating modes of a light therapy device.

### DETAILED DESCRIPTION

Various embodiments of a light therapy device and methods of use are disclosed herein. The light therapy device is configured to deliver customizable photobiomodulation therapy to meet specific therapeutic needs of an individual user. Various embodiments of the device, example components, configurations, applications, and methods of use are discussed. The example embodiments discussed herein are offered to illustrate example configurations and usages of the disclosed light therapy device. Additional examples and embodiments are possible within the scope of the disclosures herein.

### Definitions

As used herein, “light” means electromagnetic radiation having a wavelength within a range from about 280 nanometers (“nm”) to about 3.000 nm. This range includes, but is not limited to, visible light. “Visible light” refers to electromagnetic radiation having a spectrum of wavelengths from about 380 nm to about 700 nm.

As used herein “red light” means visible light having a spectrum of wavelengths within a range of about 600 nm to about 700 nm.

As used herein, “infrared light” means electromagnetic radiation having spectrum of wavelengths in a range between about 1,000 nm and about 3,000 nm.

As used herein, “near infrared light” means electromagnetic radiation having spectrum of wavelengths in a range between about 700 nm and about 1,000 nm.

As used herein, “spectral radiation” means electromagnetic radiation comprising a pluarlity of wavelengths. The plurality of wavelengths may comprise different amplitudes (i.e., luminosity) from other wavelengths in the spectrum.

As used herein, “wavelength” means about the wavelength of a peak intensity of emitted electromagnetic radiation within a larger spectrum of emitted wavelengths (i.e., spectral radiation) from a source, such as a light emitting diode (LED) chip, for example.

Embodiments of the light therapy device disclosed herein are a configured to deliver PBM light therapy through use of different operating modes. Embodiments of the light therapy device include a plurality of LED assemblies housed in a case. Each LED assembly is formed from at least one LED light source mounted on a printed circuit board (PCB) and a reflector. LED sources are semiconductor “chips” producing LED light of different spectra with constituent peak wavelengths. One or more LED sources are housed within a single reflector forming an LED assembly. In some embodiments, an LED assembly comprises two LED chips, each producing a distinct spectrum of red, near infrared (NIR), or infrared (IR) wavelengths distinct from the other LED source or sources housed by the reflector, within a spectral window.

The light therapy device can contain any number of LED assemblies in multiples of 4, in some embodiments. Some non-limiting examples include 36, 60, 72, 108, 120, 144, and 180 LED assemblies.

In some embodiments, the LED assemblies are organized into an LED array. As used herein, an array is a shape formed from a plurality of LED assemblies aligned into a repeating pattern on the PCB. In some embodiments, the array is formed by LED assemblies aligned diagonally, circularly, or in other repeating patterns.

In some embodiments, the LED assemblies are organized into modules. A module is a self-contained functional unit used in a light therapy device having a PCB boars, an LED assembly array, a cooling system, and a power supply, separate from other modules. A plurality of modules can be combined in any combination, depending on the dimensions and intended configuration of the light therapy device. For example, embodiments of the light therapy device may comprise exactly one, two, three, four, or more individual modules.

A module includes an array of LED assemblies mounted onto a printed circuit board (PCB). The module also includes a mechanism for removing heat, such as a heat sink and cooling fan, in some embodiments. In some embodiments, the module includes one or more power transformers dedicated to power constituent components, such as LED assemblies and cooling fan(s) for example, of the module.

Modules are mounted into a case. In some embodiments, the case includes a base, a front, and one or more sides. A side may be configured to mount a user interface, power cord plug, mechanical power or other switches, and the like. Cases may be physically, electrically, and communicatively coupled together in any combination, in some embodiments.

A choice of operating modes allows the user to receive a treatment with one or more spectra by activating LED sources of specific spectra, combinations of spectra, cycle duration, and the like. In some embodiments, the light therapy device can be programmed to deliver a combination of one, two, or three of red, near-infrared, and infrared spectra, alone or in various combinations, depending on selected operating mode to achieve a therapeutic goal.

The presentation and discussion of the several drawing figures which follows will describe in detail various example embodiments contemplated by this disclosure.

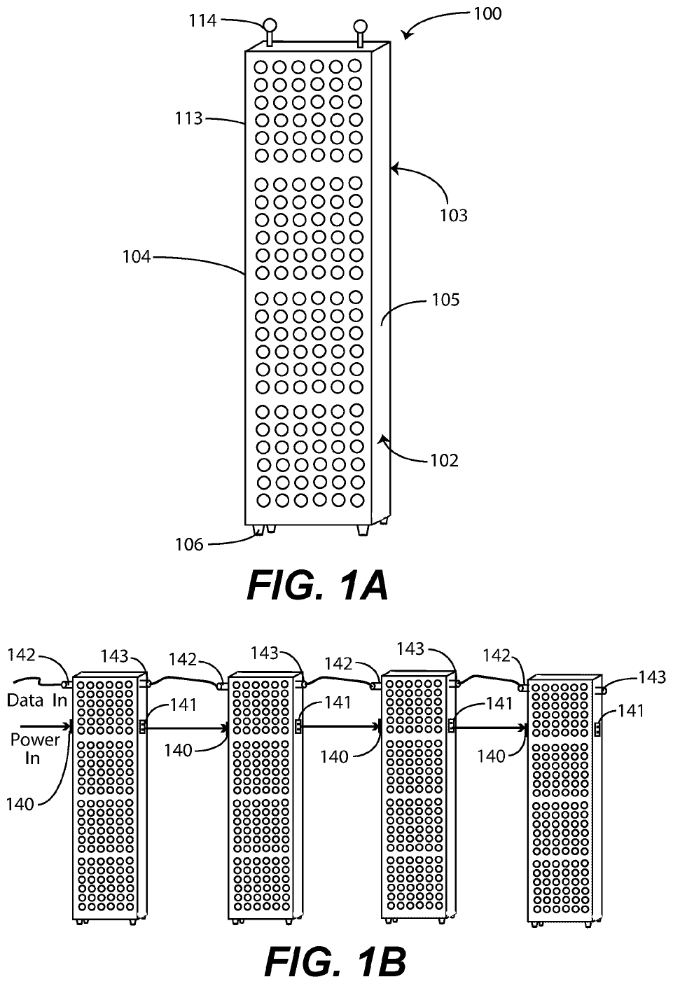

Turning to the drawing figures, FIG. 1A is a perspective view of a light therapy device 100. FIG. 1A shows light therapy device 100, including a base 103 and a front 104. Base 103 is disposed substantially parallel and opposite to front 104. In some embodiments, base 103 and front 104 are joined together by one or more sides 105 to form a case 102. Side 105 separates front 104 and base 103 of case 102.

In some embodiments, case 102 includes one or more threaded hanger bolts 114 configured to hand device 100 from a building structure wall, a door frame, or the like. In some embodiments, case 102 comprises a first surface feature (not shown) disposed on side 105 configured to interact with a second corresponding surface feature disposed on side 105 of a second device. In this fashion, multiple devices may be physically combined in a variety of sizes and dimensions to form a larger light therapy device for use in a specific building space.

Case 102 is configured to contain and mount one or more LED array modules 120 within an interior space bounded and defined by base 103, front 104, and side 105. Materials used to form case 102 include sheet steel, aluminum, and other suitable metals and metal alloys used in the manufacture of cases and cabinets for electrical devices. Heat-resistant plastics, such as used in automotive and aerospace applications, may also be used, such as Acetal (polyformaldehyde), Kynar® (polyvinylidene fluoride), PEEK (polyetheretherketone), for example. ABS and other plastics lacking heat and electrical resistance properties would not be suitable.

As shown in FIG. 1A, front 104 comprises as plurality of openings 113 through which the LED assemblies are aligned therewith. The specific configuration of openings 113 in front 104 corresponds with the LED assembly array pattern, therefore the openings may be formed in rows and columns (as shown in FIGS. 1A-B), or other configurations, without limitation. In some embodiment, an inside surface of base 103 (not shown) is configured with a mounting feature for mounting a printed circuit board 112 bearing a plurality of LED assemblies 111.

Case 102 can take a variety of forms and configurations, including a panel, as shown in FIG. 1A. One or more panels may be mounted within an enclosure, such as a booth. Two or more panels may be joined together by interacting surface features disposed on corresponding sides 103 between the one or more panels, in some embodiments. A panel may be flat, as shown in FIG. 1, but this is not intended to be limiting. In some embodiments, the panel is curvilinear. A curvilinear panel may be incorporated into an enclosure wherein the user is recumbent, similar to a tanning bed. In some embodiments, case 102 includes one or more feet 106. In some embodiments, case 102 includes a stand configured to allow the user to position device 100 on a table, floor, or other flat surface. The stand may be configured to position device 100 at an angle, which may be adjustable to optimize the angle of electromatic radiation from device 100 incident to a body surface of the user. In some embodiments, case 102 is configured to be mounted on a surface within a building or a dwelling, such as a wall or a ceiling. In some embodiments, case 102 is configured to me mounted to or housed within an item of furniture, such as a headboard for a bed, for example.

In some embodiments (not shown), case 102 is formed from a flexible material, such as a soft plastic, a fabric, a textile, or the like. Use of the flexible material enables light therapy device 100 to be configured into a wearable article, such as a belt worn around the chest or abdomen next to the skin of a user. The wearable article may also be a garment, such as a shirt or vest. In these and some other embodiments, side 105 is formed from a strip of material coupled to front 104 and base 103. In some embodiments, side 105 is formed from an extension or reflection of material forming front 104 or base 103.

FIG. 1B is a diagram of multiple light therapy devices electrically and communicatively coupled together. FIG. 1B shows four separate devices 100 having a common power source (“Power In”). Light therapy device 100, in some embodiments, is powered by an external power source, such as a 110 or 120-volt A/C household power supply outlet (not shown) delivering at least about 360 Watts of power. In some embodiments, multiple devices 100 can be electrically coupled together in series or “daisy chained” by connecting a power out connector 141 on a first device 100 to a power in connector 140 in a second device 100, as shown in FIG. 1B. Either or both power in connector 141 or power out connector 141 are mounted on side 105, base 103, or front 104 of case 102, in some embodiments. In some embodiments, light therapy device 100 includes a data in port 142 and a data out port 143 mounted on case 102, as also shown in FIG. 1B. In some embodiments, data in port 142, data out port 143, or both port 142 and port 143 are standard telephone jacks. In some embodiments, either or both ports 142 and 143 may be ethernet connector jacks, coaxial cable jacks, or other wired data communication jacks. In some embodiments, either or both ports 142 and 143 are mounted on base 103 or front 104 of case 102.

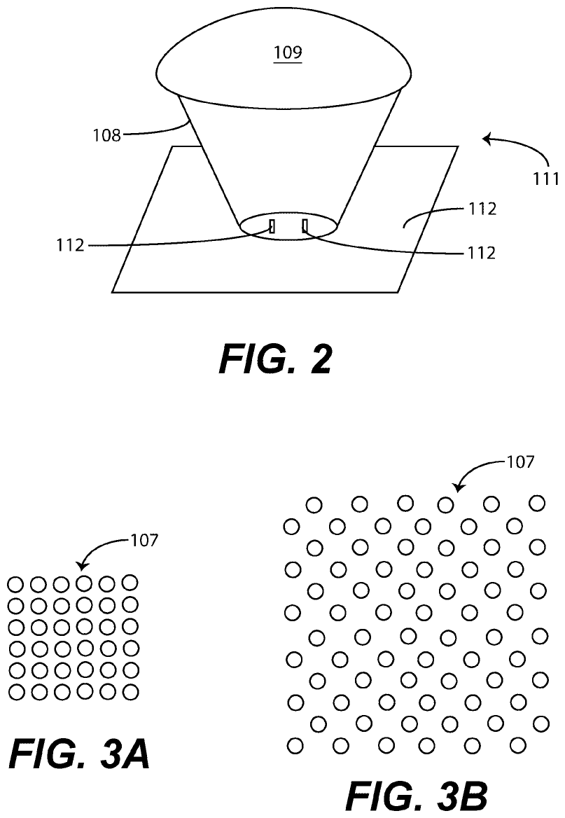

FIG. 2 is a perspective-view diagram of an LED assembly 111. As shown in FIG. 2, LED assembly 111 includes one or more LED chips 110 mounted on PCB 112. An LED chip is a semiconductor device, known in the art, that produces light of a given spectrum, having a peak wavelength, or “color” across a range of wavelengths, when powered with a current source. In some embodiments, LED assembly 111 comprises exactly two (2) LED chips 110 per each assembly 111 comprised by light therapy device 100. During any of the several specific operating modes, discussed herein as examples, a first LED chip 110 in one assembly 111 may be powered (“on”) concurrent with a second LED chip 111 in the same assembly not receiving power (“off”), in a given operating mode. This arrangement creates increased functionality of device 111 compared to existing light therapy devices wherein a pluarlity of operating modes, discussed herein, are possible.

In the embodiments discussed herein, only one, single LED chip 110 of a single assembly 111 will receive power (“on”) at any time. For example, in an LED assembly 111 having a first LED chip 110 of one color and a second LED chip 110 of a second color, either color will be “on” and the other color “off,” or both colors will be “off,” according to the operating mode selected by the user. PCB board 112 is configured, in some embodiments, to power each individual LED chip 110, not each individual LED assembly 111, such powering an assembly 111 does not require that all of a plurality of LED chips 110 comprised by the assembly 111 be “on” or “off” at the same time.

In some embodiments, the two LED chips 110 per assembly 111 include two different colors alternating with adjoining assemblies 111 having LED chips 110 of two additional different colors; i.e., four different (distinct spectra with four different peak wavelengths-four colors disposed two colors at a time per assembly 111 across two adjoining assemblies 111) LED chip colors comprised by light therapy device 100. In some embodiments, there are an equal number of each color of LED chips 100 in a single light therapy device 100, although this is not intended to be limiting. In some embodiments, there are unequal numbers of LED chips 110 of two, three, four, or more separate colors. In embodiments of light therapy device 100 having equal numbers of LED chip color types, the relative numbers of the different LED chip 110 types are expressed throughout the disclosures herein as percentages (i.e., 25% first color; 25% second color; 25% third color; and 25% fourth color) or ratios (i.e., 1:1:1:1). In some embodiments of light therapy device 100 having colors of LED chips 110 in unequal numbers, the relative numbers may be expressed to reflect the inequality; i.e., 25%; 25%; 0; 50% or 1:1:0:2, for example, and so on. The relative numbers of chip colors need not equal the number of LED assemblies 111 in embodiments having more than one color LED chip 110 per assembly 111.

A reflector 108 is coupled to LED chip 110. In some embodiments, a lens 109 (as FIG. 2 shows aligned with reflector 108 absent case 102), is mounted to an opening 113 in front 104 of case 102 and aligns with or is coupled to reflector 108. Reflector 108, in some embodiments, is a frustoconical body having a reflectorized inner surface bounding a space extending between LED chip 110 and opening 113. Reflector 108 is configured to constrain the electromagnetic radiation generated by LED chip 110 within the space and to reflect the electromagnetic radiation through opening 113. Materials used to form reflector 108 include any relatively lightweight, heat-resistant plastic with a reflectorized inner surface, known in the electronic device manufacturing arts.

Lens 106 may or may not be included as a component of LED assembly 111, depending on the particular embodiment of light therapy device 100. Consequently, in some embodiments of light therapy device 100 having a mix of multiple LED chip spectra contained within LED array 107, any LED assembly 111 may not include lens 109. Some embodiments of light therapy device 100 will have a lens 109 coupled to all LED assemblies 111. In other non-limiting examples, one-half of the total number of LED assemblies 111 in an LED array 107 will include lens 109. In some embodiments, one-quarter of the total number of LED assemblies 111 in an LED array 107 will include lens 109. In some embodiments, every other LED assembly 111 in a horizontal row of LED array 107 will include lens 109. In some embodiments, every third LED assembly 111 in a horizontal row of LED array 107 will include lens 109. In some embodiments, every fourth LED assembly 111 in a horizontal row of LED array 107 will include lens 109. And so on.

FIGS. 3A-B are diagrams LED arrays. FIGS. 3A-B show two examples of possible configurations of an LED array 107. As seen in FIG. 3A, array 107 may be formed from a plurality of LED assemblies 111 aligned into both horizontal rows and vertical columns. As shown in FIG. 3B, and in some other embodiments, array 107 is formed from LED assemblies 111 aligned horizontally with alternating rows staggered vertically. The array 107 configurations shown in FIGS. 3A-B are by example only. Other configurations of LED assemblies 111 into LED arrays 107 are contemplated by the disclosures herein.

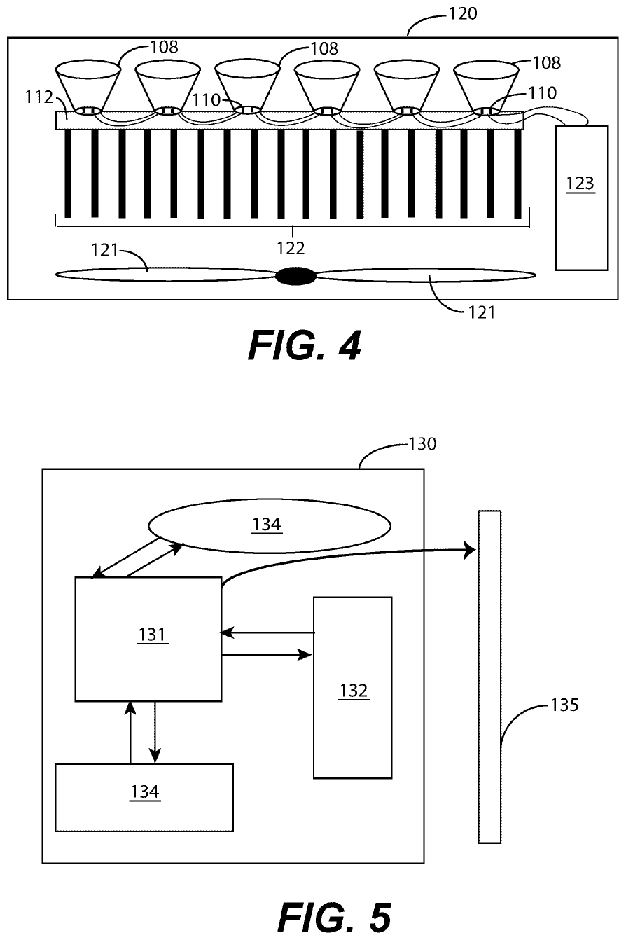

FIG. 4 is a diagram of a module used in some embodiments of a light therapy device. FIG. 4 shows module 120, having, in some embodiments, LED array 107 mounted on a single PCB 112 thermally coupled to a heat sink 122. In some embodiments, heat sink 122 is thermally coupled to PCB 112 through a common mounting of heat sink 122 to an aluminum mounting plate within the interior of case 102 with PCB 112. Heat sink 122 is formed from a material with a high thermal inertia, typically aluminum, for example, into a series of fin-like elements to maximize surface area for heat dissipation. Heat sinks for PCB boards and other elements of an electronic device are widely available and known in the art. In some embodiments, a cooling fan 121 is configured to circulate air through and around heat sink 122. In some embodiments, cooling fan 121 is mounted to base 103 over a ventilation aperture (not shown) in base 103 generally corresponding in size to the size and blade-span diameter of fan 121. Cooling fan 121 operates to draw outside air into case 102 through the ventilation aperture, blowing the cool outside air across heat sink 122 and out of case through holes (not shown) formed within side 105. In this manner, heat generated by electrical resistance and infrared radiation spectra from LED chips 110, a transformer 123 (powering LED chips 110 and discussed below), and other electronic components is vented from case 102. Any number of commercially available fans used in electronic devices known and used in the desktop computer and electronics art may be used as cooling fan 121.

In some embodiments of device 100 having a plurality of modules 120 and a corresponding plurality of cooling fans 121, a power source electrically separate from transformer 123 (driver providing power to LED chips 110 via PCB 112) is desirable. An example is a commercially available 12V DC cooling fan power supply such as used in desktop computers. One or more cooling fan power supplies are used depending on the number of modules 120 and the number of cooling fans 121 per module 120 used in light therapy device 120.

In some embodiments, module 120 includes a dedicated transformer 123 electrically coupled to PCB 112 to power the pluarlity of LED chips 110 on PCB 112. A commercially available LED driver, such as a 60 W, 22.5V LED driver, is one non-limiting example of transformer 123. In some embodiments, a plurality of cooling fans 121 cooling a single module 120 or a plurality of separate modules 120 are powered from the same power source separate from transformer 123.

Module 120 is a functional unit of light therapy device 100, in some embodiments. Embodiments of light therapy device 100 may include one or a plurality of modules 120. In some embodiments, device 100 includes exactly two (2) modules 120. In some embodiments, device 100 includes exactly four (4) modules 120. In some embodiments, device 100 includes exactly six (6) modules 120).

A plurality of modules 120 may be mounted generally co-planar to case 102, as shown in FIG. 1, in some embodiments. In some other embodiments (not shown), a plurality of modules 120 may be arranged at angles to other modules 120 within the same light therapy device 100, such as on two or more light therapy device panels mounted inside an enclosure, such as a therapy booth, or mounted to adjoining walls of a building structure.

### Operating Modes:

Embodiments of light therapy device 100 may be configured to deliver different combinations of light spectra to the user, enabling delivery of many distinct therapies using a single device 100. The user may desire treatment using light within a NIR spectral band to derive a specific therapeutic benefit, and light from a visible red-light band to derive a different specific therapeutic benefit, for example. In some instances, the user may seek therapy with two different peak spectra of NIR radiation in the same therapy session. In some instances, the user may wish to incorporate red light of a shorter wavelength outside of the NIR spectrum with the NIR spectrum using a single device 100. In some embodiments, the user may wish to incorporate and IR spectrum with either or both of IR and red light spectra. Accordingly, some embodiments of light therapy device 100 are configured to deliver a selection of operating modes 126 to the user, based on the user's therapeutic needs.

Table 1 below details an example of the possible operating modes 126 in some embodiments of a light therapy device 100 having LED arrays 107 using different combinations of LED assemblies. The embodiments listed in Table 1 incorporate LED assemblies of four (4) example wavelengths: 630 nm; 660 nm; 810 nm; and 850 nm. In some embodiments, an LED chip 110 emitting a peak spectra centered at about 940 nm is substituted for the LED chip 110 peak spectra centered at about 850 nm listed in the table. Reasons for this and other substitutions, for example, include evidence that exposure to certain wavelengths of infrared light, including about 940 nm, immediately prior to use of a tanning bed or similar tanning enclosure may be partially protective against sunburn after subsequent exposure to ultraviolet light. The “Chip Wavelength” column in Table 1 lists the approximate peak spectral wavelength of peak intensity within the spectra generated by any given LED chip 110, and the “Percentage” column lists the percentage of total LED Assemblies 111 of device 100 that are illuminated during treatment with the listed operating mode.

TABLE 1

| | LED CHIP PEAK | |
|---|---|---|
| | SPECTRAL | |
| MODE | WAVELENGTH | PERCENTAGE |
| 1 - Red/NIR | 630 nm | 25% |
| | 660 nm | 25% |
| | 810 nm | 25% |
| | 850 nm | 25% |
| 2 - Red Only | 630 nm | 50% |
| | 660 nm | 50% |
| | 810 nm | OFF |
| | 850 nm | OFF |
| 3 - 660/NIR | 630 nm | OFF |
| | 660 nm | 50% |
| | 810 nm | 25% |
| | 850 nm | 25% |
| 4 - 630/NIR | 630 nm | 50% |
| | 660 nm | OFF |
| | 810 nm | 25% |
| | 850 nm | 25% |
| 5 - 850/Red | 630 nm | 25% |
| | 660 nm | 25% |
| | 810 nm | OFF |
| | 850 nm | 50% |
| 6 - 810/Red | 630 nm | 25% |
| | 660 nm | 25% |
| | 810 nm | 50% |
| | 850 nm | -OFF- |
| 7 - 660/Red Only | 630 nm | OFF |
| | 660 nm | 50% |
| | 810 nm | OFF |
| | 850 nm | OFF |
| 8 - 630/Red Only | 630 nm | 50% |
| | 660 nm | OFF |
| | 810 nm | OFF |
| | 850 nm | OFF |
| 9 - 850 Only | 630 nm | OFF |
| | 660 nm | OFF |
| | 810 nm | OFF |
| | 850 nm | 50% |
| 10 - 810 Only | 630 nm | OFF |
| | 660 nm | OFF |
| | 810 nm | 50% |
| | 850 nm | OFF |
| 11 - NIR Only | 630 nm | OFF |
| | 660 nm | OFF |
| | 810 nm | 50% |
| | 850 nm | 50% |

In some embodiments, device 100 contains two LED chips 110 having different wavelength spectra per LED assembly 111 and is electronically configured to allow for eleven (11) distinct modes to be used. In some embodiments, device 100 contains two LED chips 110 colors per LED assembly 111 and is configured to allow for seven (7) distinct modes to be used. This is by example only. Additional combinations of chip wavelength spectra per LED array 107 are possible and multiple distinct modes are within the scope of the disclosed invention.

In some example embodiments, operating modes may include activation of one or more LED chips 110 having an output of at least about 1,000 milliwatts per nanometer (mW/nm) at about 630 nm and about 670 nm simultaneously from each LED chip 110.

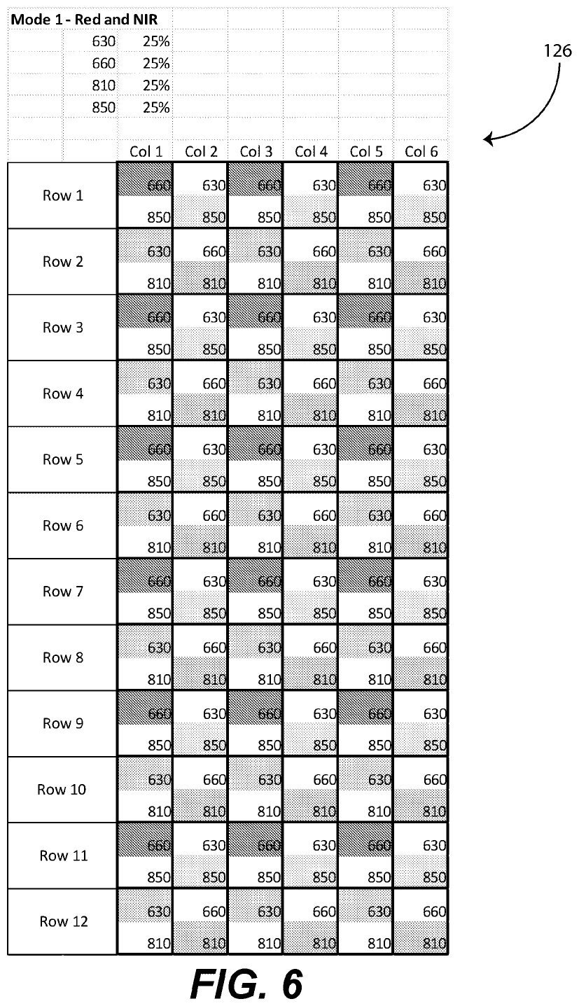

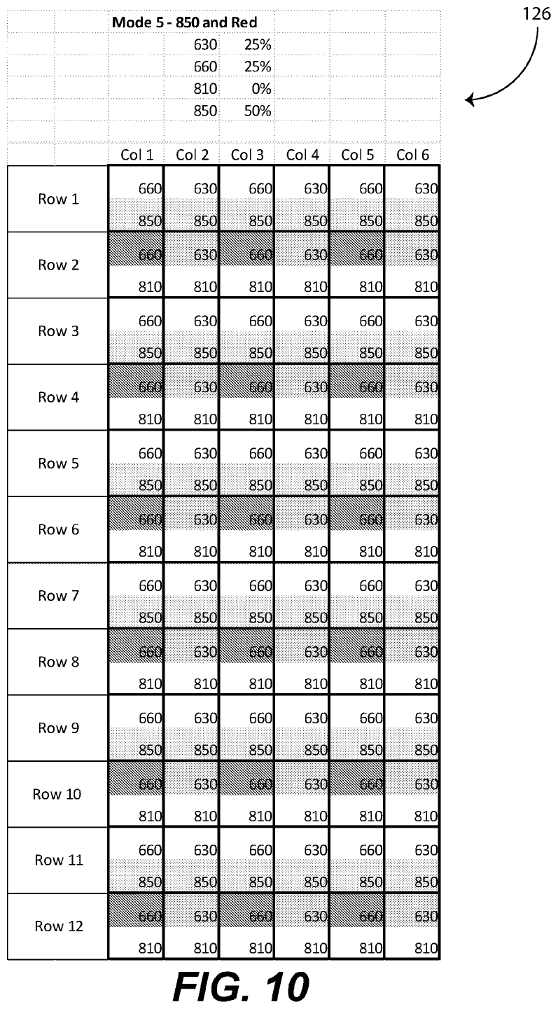

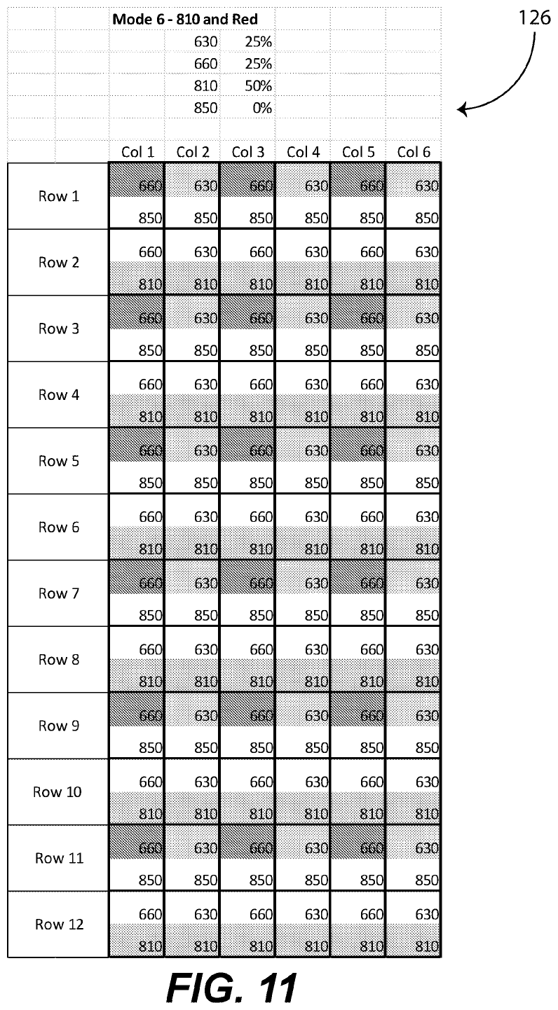

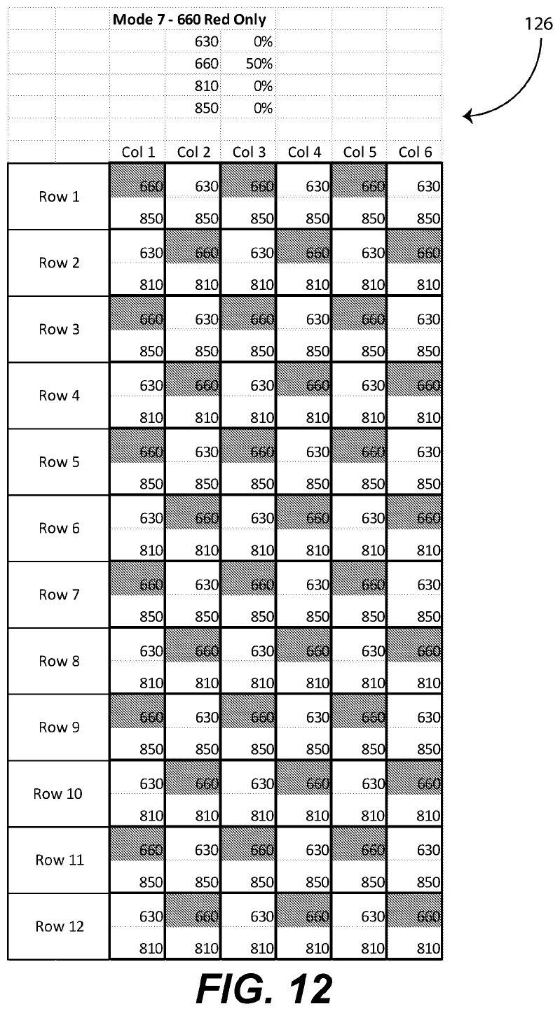

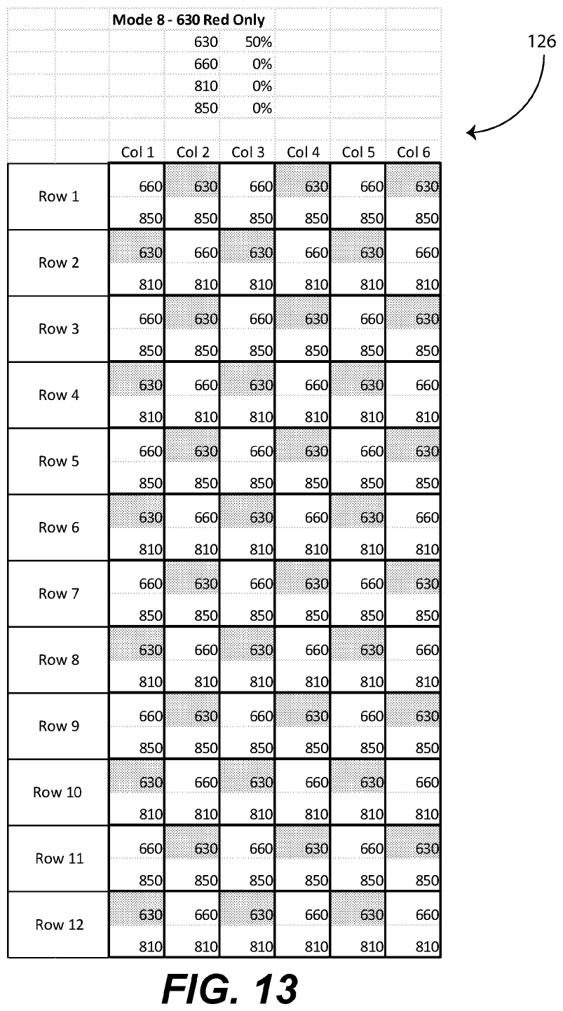

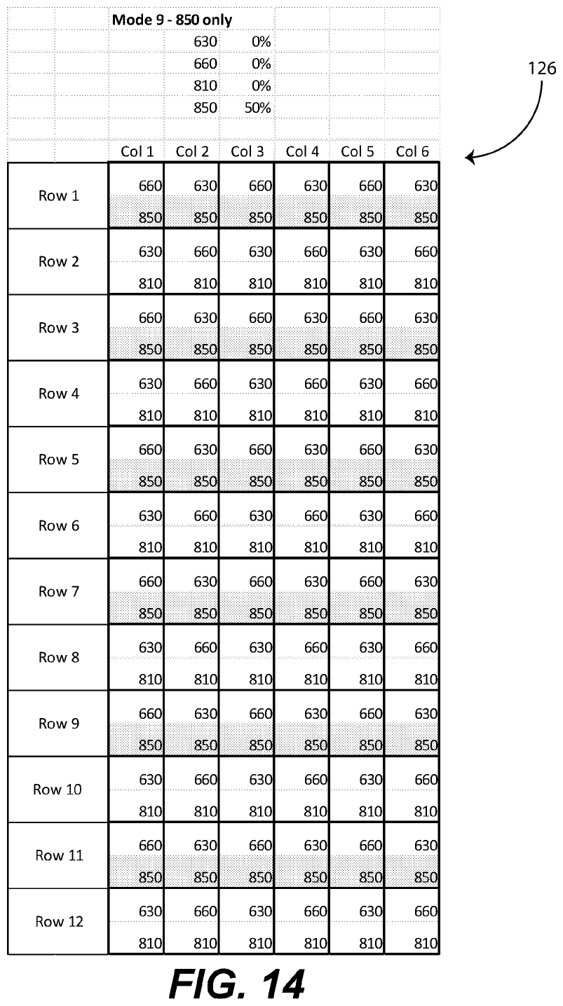

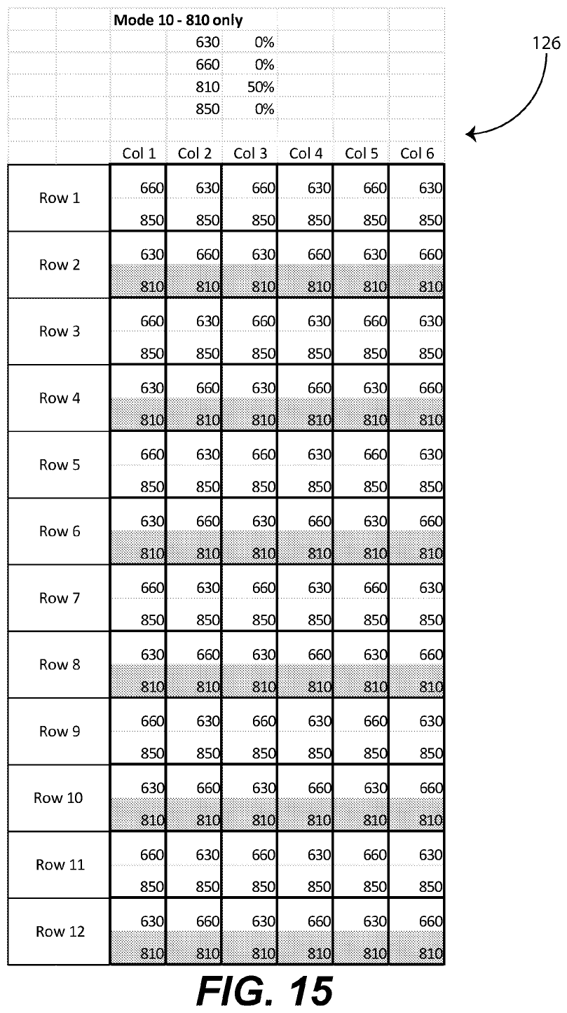

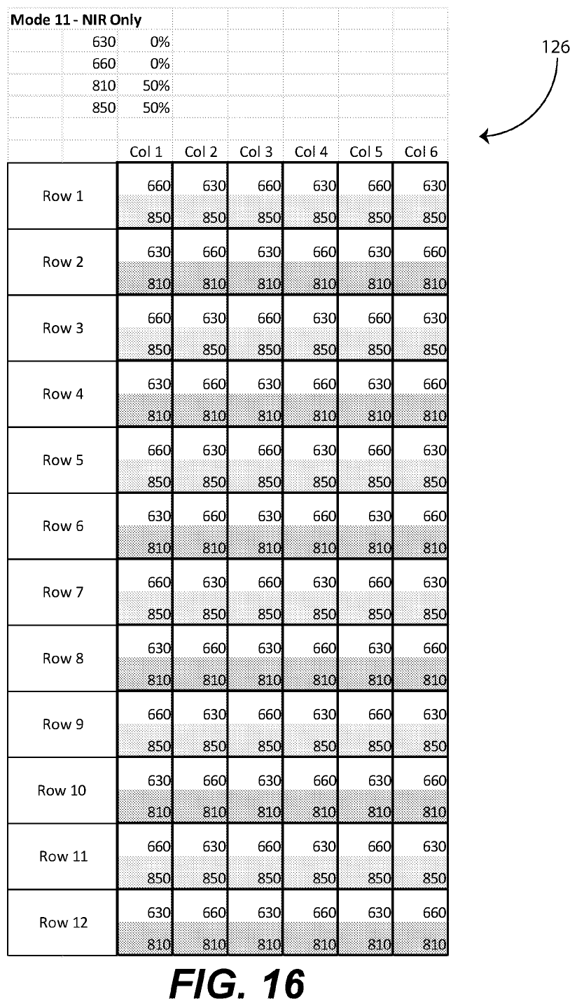

FIGS. 6-16 are charts of different examples of LED assembly 111 activation wavelengths and combinations in some example embodiments of operating modes 126 of light therapy device 100. Note that in the examples depicted in the several drawing figures, each LED assembly 111 comprises two (2) wavelengths: i.e., Row 1/Col. 1 of FIG. 16 comprises a 660 nm LED chip 110 and an 850 nm LED chip 110. FIGS. 6-16 show examples of different operating modes of LED array 107 comprising twelve rows of six columns of LED assemblies 111 configured such that each assembly 111 aligns with a corresponding assembly 111 in any adjoining row and also in any adjoining column. In some embodiments, two (2) modules 120 form the 12-row by six-column example array 107 depicted in the Figures. Each Figure depicts a different operating mode 126 that is distinct from modes 126 shown in the other Figures. Shading in the block indicates the LED chip 110 having the indicated wavelength is powered in the particular mode 126 shown by the Figure.

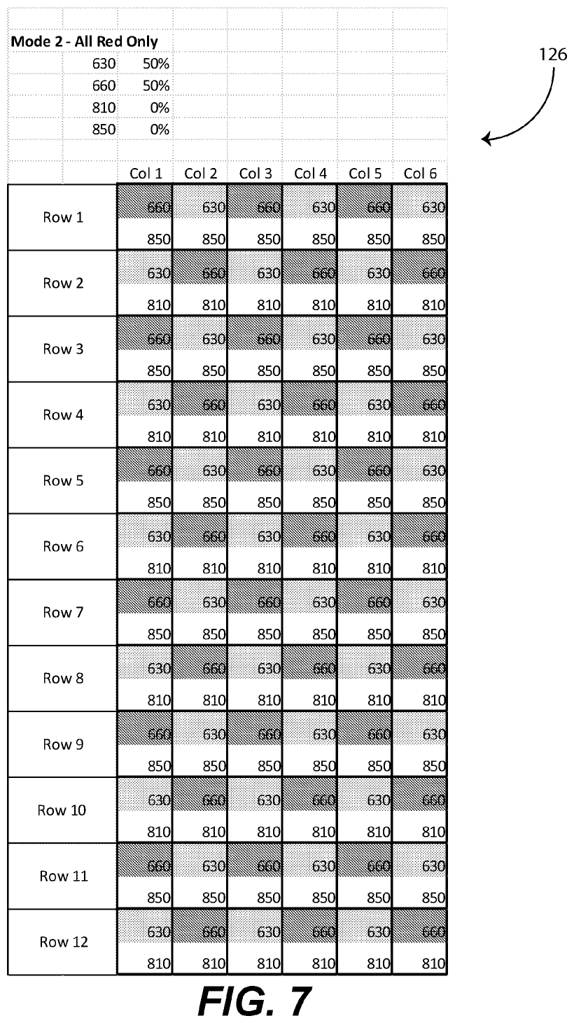

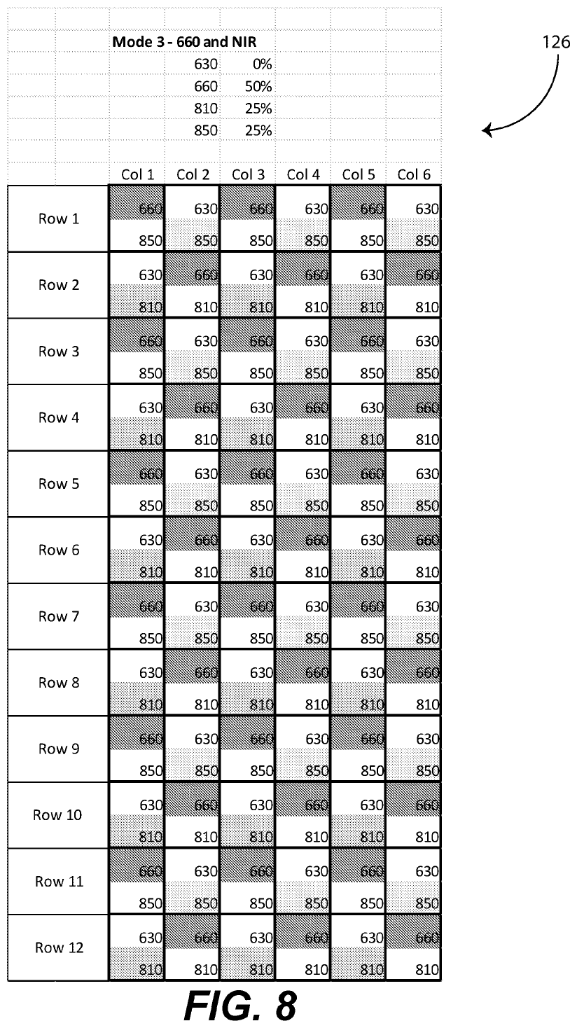

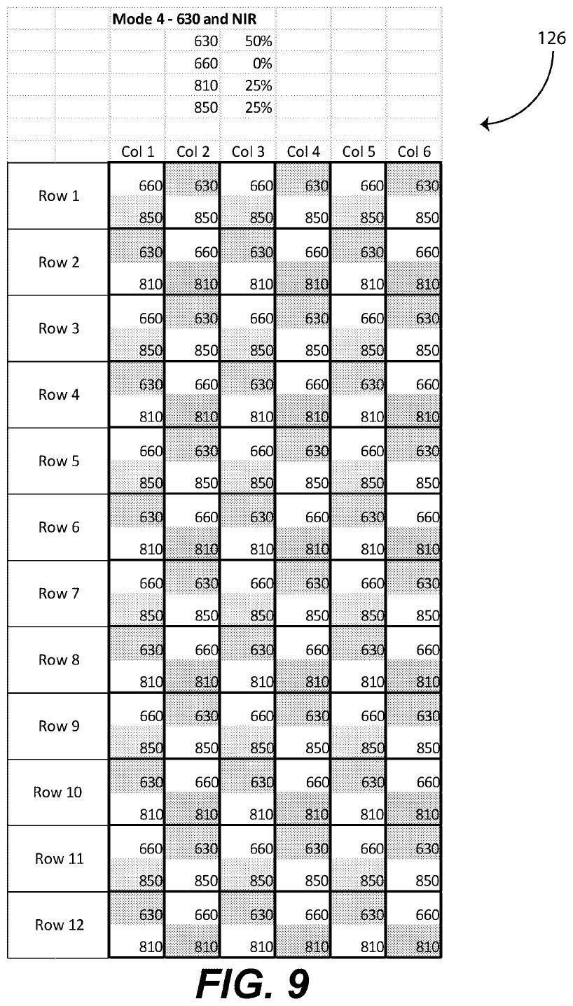

As shown by FIGS. 6-16, in some embodiments, all LED chips 110 in any shaded (not white) box are illuminated in the mode for the entire treatment duration wherein light therapy device 100 is operating the specific mode 126 depicted in the Figure. FIGS. 6-16 show eleven distinct operating modes 126 numbered 1-11. For example, FIG. 6 shows operating mode 126 #1, FIG. 7 shows operating mode 126 #2, FIG. 8 shows operating mode 126 #3, and so on through FIG. 16 that shows operating mode 126 #11. In some embodiments, device 100 cycles through a plurality of distinct operating modes 126 consecutively at a set interval, For example, in some embodiments, the user may set device 100 to sequentially cycle through operating mode 126(#3), mode 126(#7) and mode 126(#10) at one-minute increments. In some embodiments, the increments are longer than one minute, such as ninety seconds, two minutes, five minutes, etc. In some embodiments, the increments are of and same length. In some embodiments, the increments are of different lengths, in any combination. In some embodiments, upon completing one full cycle, device 100 cycles through the pluarlity of modes 126 selected by the user one or more additional times. As described in this paragraph, “duration” is a first parameter of operating mode 126. Additional parameters (i.e., second parameter, third parameter, etc.) include brightness, intensity, etc., as described in the examples below.

Although Table 1 and FIGS. 6-16 describe operating modes 126 in terms of the relative percentages of powered LED chips 110 of a given peak spectral wavelength, these percentages may alternatively be expressed as a ratio. For example, operating mode 126 (#1) comprises powering chips 110 with peak wavelength intensities of 630 nm, 660 nm, 810 nm, and 850 nm powered at a ration of 1:1:1:1: mode 126 (#2) of 1:1:0:0; mode 126 (#3) of 0:2:1:1; mode 126 (#4) of 2:0:1:1; mode 126 (#5) of 1:1:0:2; mode 126 (#6) of 1:1:2:0: mode 126 (#7) of 0:2:0:0; mode 126 (#8) of 2:0:0:0; mode 126 (#9) of 0:0:0:2; mode 126 (#10) of 0:0:2:0; and mode 126 (#11) of 0:0:2:2.

In some embodiments, mode 126 is configured to vary a second parameter in addition to duration. In some embodiments, a second parameter is brightness of chip 100. In some embodiments, LED chips 110 having a same first wavelength are illuminated at a first brightness and LED chips 110 having a same second wavelength are illuminated at a second brightness. In some embodiments, additional parameters may be incorporated into operating mode 126. In some embodiments, programming different parameters, such as intensity and duration, in different combinations allows the use to simulate the changing solar spectra during sunrise and sunset. In this example, such therapy may aid the user in waking up in the morning. Alternatively, operating mode 126 may be set to help the user “wind down” at day's end, in some embodiment, promoting a more restful, healthy night's sleep. By varying the value of a first parameter, a second parameter, and so on, in different combinations, the user can enable a high level of light therapy customization from device 100.

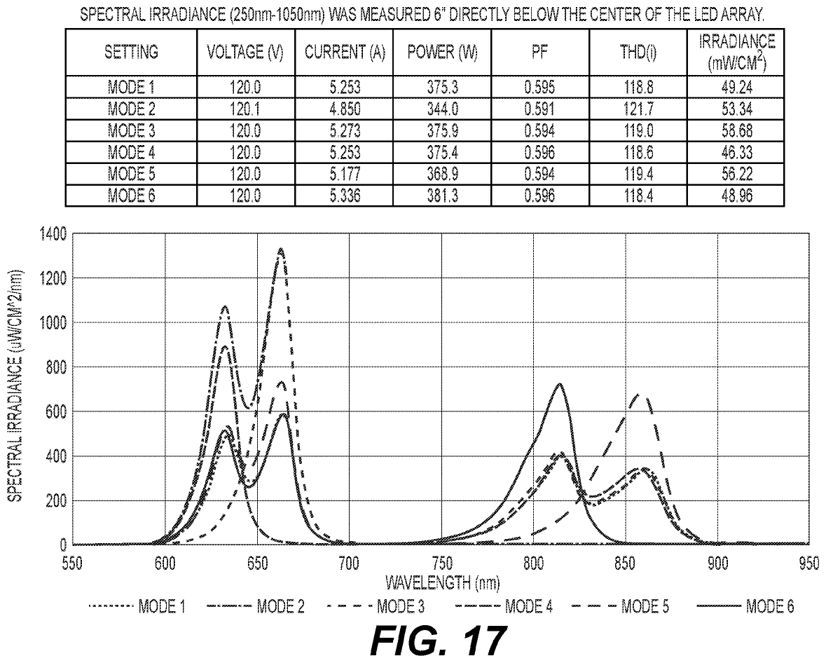

A clearer understanding of different operating modes 126 outlined in FIGS. 6-16 is illustrated by a review of the spectra of a plurality of modes 126 superimposed on a single graph. Accordingly, FIG. 17 is a graph of irradiance plotted against wavelength for example spectra utilized in six (6) separate operating modes of a light therapy device. FIG. 17 shows example spectra of a plurality of operating modes 126(#s 1-6 from FIGS. 6-16). Six separate curves are shown on this graph, each curve representing a single operating mode 126. Each curve has four peaks, representing the listed peak wavelengths of (about) 630 nm, 660 nm, 810 nm, and 850 nm. The relative luminosity (i.e., curve height on the y-axis “Spectral Irradiance”) reflects the ratio (listed as a percentage in FIGS. 6-16) of the number of activated LED chips 110 of the listed wavelength to the number of LED chips 110 of the other three wavelengths for the particular operating mode 126 (#1, #2, . . . #6) represented by the curve. As can be seen by examining the curves of FIG. 17, different operating modes 126 have distinct luminescence levels across the red light-NIR-IR spectra, ranging from about 580 nm through about 900 nm, in these examples and in some embodiments. Thus, a particular operating mode 126 may be selected by the user to emphasize light therapy at a certain wavelength, a certain intensity at one wavelength relative to other wavelengths, etc. In addition to changing the intensity (luminescence) around a specific wavelength, additional parameters such as duration and others as discussed herein, can be selected by the user, in some embodiments of light therapy device 100.

For example, in some embodiments, light therapy device 100 includes a computing module 110 (shown in FIG. 5 and discussed in detail herein below) programmed with software code to execute an algorithm. An algorithm includes instructions stored in a memory 132 and used by a processor 131 to execute the one or more operating modes 126. For example, in some embodiments, an algorithm, via computing module 110, powers array 107 (or plurality of arrays 107) of LED chips 110 to provide changing exposure to light spectra during the course of a day. At sunrise, the spectral ratio of longer IR wavelengths to shorter red wavelengths is different than at midday, which is also different than at sunset. Therefore, light therapy device 100, in some embodiments, will execute programed algorithms causing the light therapy device to deliver a first, early morning spectral ratio, later switching to a midday spectral ratio, and then completing the treatment session with a sunset/evening spectral ratio. The “spectral ratio” created by the selected mode 126 can be initially selected, changed, or otherwise modified as desired to address a specific therapeutic goal, such as improving sleep quality, enhancing muscle recovery after exercise or resistance training, increasing alertness or focus, etc. Thus, light therapy device 100 maximizes red light, NIR, and IR PBM preconditioning and repair benefits, in some embodiments. Device 100 is configured, computing module 130, to add, delete, modify, and execute a pluarlity of treatment algorithms. The aforementioned descriptions are offered by way of example only and are not intended to be limiting.

### Controls:

Some embodiments of device 100 include a computing module for processing control commands, along with monitoring the functionality of device 100. Additionally, a user interface is provided for controlling different aspects of the operation of light therapy device 100, regardless of whether the embodiment includes a computing module. The user interface enables the user to execute commands controlling power, operating mode 126, duration of therapy, time (of day) of therapy, and other functions. The user interface may take the form of a conventional mechanical power switch, a touch panel, a wireless control, or some combination of the above.

FIG. 5 is a diagram of computing module 130. FIG. 5 shows computing module 130 having a processor 131, a memory 132, a communications interface 133, and a user interface 134. Only some embodiments of device 100 include a computing module 130. Arrow-tipped lines in FIG. 5 denote the flow of information between the various components of computing module 130 and a display 135.

User interface 124, in some embodiments, is a touch-control panel mounted on case 102. The control panel may comprise mechanical switches, pressure switches, a touchscreen or other control interface elements. The user may access and select the various operating modes 126 directly from the user interface.

Processor 131, in some embodiments, is a commercially available microprocessor widely used in consumer electronic device control systems and known in the art. Processor 131 executes instructions received directly from user interface 134, according to software stored in memory 132, or from user interface 134 and memory 132 in combination. Memory 132 includes any combination of random access (cache) memory (RAM) and non-RAM “main” memory, in some embodiments.

In some embodiments, display 135 is physically located on the control panel or other user interface 133. Display 135, in some embodiments, shows the specific mode 126 selected by the user and executed by computing module 130. For example, the user may select “Mode 11” (as shown in Table 1 herein above) through user interface 133, whereupon display 135 will show “810 nm-850 nm.” Display 135 can take different forms depending on the embodiment of therapy device 100. In some embodiments, display 135 is a simple, conventional light-emitting diode (LED) or liquid crystal display (LCD) panel mounted on case 102. In some embodiments, display 135 is a computer monitor screen communicatively coupled to processor 131 by a wired or a wireless connection. In some embodiments, display 135 is a touchscreen on a smartphone or other handheld device. In some embodiments, display 135 is a handheld display and user interface 134 combined into a remote handheld device. Examples of a remote handheld device may include a remote control, smartphone, tablet, other handheld computing device, bearing an application or “app,” or the like. The handheld device may communicate with processor 131 via a wired connection or a wireless connection, in some embodiments. Non-limiting examples of a wireless connection include Bluetooth, Wi-Fi network, or the like.

In some embodiments, software stored on memory 132 comprises code controlling power supply to PCB 112 and LED chips 110 wherein some chips 110 receive power and other chips 110 are not powered. In some embodiments, chips 110 are powered on and off in a sequence according to the particular operating mode 126 selected by the user. In some embodiments, software stored on memory 132 includes one or more algorithms to direct the specific operating mode 126 selected by the user. Additional software may be installed on an additional memory, or “second memory” incorporated into a remote computing device to enable wireless communication with computing module 130 via Bluetooth or other wireless platform. In some embodiments, data on usage such as duration, frequency specific, and similar data specific to mode 126 used and cumulative user exposure to different light spectra resides. Data regarding cumulative exposure times and amounts is useful to protect user safety, in some embodiments.

In some embodiments, software code residing on memory 132 may exclude accessibility of certain modes 126 for user safety. The user may activate or de-activate the exclusion criteria via user interface 132, as desired. One example of such exclusion are one or more operating modes 126 containing entirely near infrared (“NIR”) light. A non-limiting example of this is (operating mode 126) “Mode 11” shown in Table 1 herein above. This and other safety features are desirable, in some embodiments, because repeated, high levels of exposure to infrared light has been shown to induce cataract-like changes in the lens of the eye of humans and other mammals.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings herein above.

1. A light therapy device comprising:
a case having an interior defined by at least one side and configured to mount a plurality of light emitting diode (LED) assemblies within the interior, wherein each LED assembly comprises exactly two LED chips having different emission spectra mounted on a printed circuit board and a reflector, wherein the plurality of LED assemblies are arranged in a repeating pattern of rows and columns forming an array, and
wherein the array comprises repeating pairs of two (2) LED assemblies, each pair of LED assemblies emitting exactly four (4) different LED chip emission spectra.

2. The light therapy device of claim 1, wherein each row of the array is arranged with an LED assembly having a combination of two different emission spectra LED chips alternating with an adjoining LED assembly with two LED chips having a different emission spectra combination.

3. The light therapy device of claim 1 having an exactly equal number of each LED chips with the same emission spectra in the light therapy device.

4. The light therapy device of claim 1, wherein fifty percent (50%) of the plurality of LED assemblies comprise an LED chip having a peak intensity wavelength of about 810 nm.

5. The light therapy device of claim 1, wherein fifty percent (50%) of the plurality of LED assemblies comprise an LED chip having a peak intensity wavelength of about 850 nm.

6. The light therapy device of claim 1, wherein fifty percent (50%) of the plurality of LED assemblies comprise an LED chip having a peak intensity wavelength of about 940 nm.

7. The light therapy device of claim 1, further comprising a computing module having at least one software algorithm residing on a memory, wherein the at least one software algorithm causes the light therapy device to execute at least one operating mode.

8. The light therapy device of claim 7 configured to execute the at least one operating mode in response to instructions received from a wireless remote handheld device having a user interface.

9. (canceled)

10. (canceled)

11. (canceled)

12. (canceled)

13. A method of operating the light therapy device of claim 1 comprising a step powering “on” only one LED chip of each of the LED assemblies emitting the exactly two (2) different emission spectra while the remaining LED chip of the same LED assembly is not powered on.

14. The method of claim 13, wherein the light therapy device comprises a number of LED assemblies having an LED chip with a peak emission spectra of about 660 nm and an LED chip with a peak emission spectra of about 850 nm together in the same LED assembly.

15. The method of claim 13, wherein the light therapy device comprises a number of LED assemblies having an LED chip with a peak emission spectra of about 630 nm and an LED chip with a peak emission spectra of about 850 nm together in the same LED assembly.

16. The method of claim 13, wherein the light therapy device comprises a number of LED assemblies having an LED chip with a peak emission spectra of about 630 nm and an LED chip with a peak emission spectra of about 810 nm together in the same LED assembly.

17. The method of claim 13, wherein the light therapy device comprises a number of LED assemblies having an LED chip with a peak emission spectra of about 660 nm and an LED chip with a peak emission spectra of about 850 nm together in the same LED assembly.

18. The method of claim 13, wherein the light therapy device comprises a number of LED assemblies having an LED chip with a peak emission spectra of about 660 nm and an LED chip with a peak emission spectra of about 810 nm together in the same LED assembly.

19. The method of claim 13, wherein the light therapy device comprises:
exactly 25% of the total LED chips having a peak emission spectra of about 630 nm;
exactly 25% of the total LED chips having a peak emission spectra of about 660 nm;
exactly 25% of the total LED chips having a peak emission spectra of about 810 nm; and
exactly 25% of the total LED chips having a peak emission spectra of about 850 nm.

20. The method of claim 13, wherein the light therapy device comprises:
exactly 50% of the total LED chips having a peak emission spectra of about 630 nm; and
exactly 50% of the total LED chips having a peak emission spectra of about 660 nm.

21. The method of claim 13, wherein the light therapy device comprises:
exactly 50% of the total LED chips having a peak emission spectra of about 660 nm;
exactly 25% of the total LED chips having a peak emission spectra of about 810 nm; and
exactly 25% of the total LED chips having a peak emission spectra of about 850 nm.

22. The method of claim 13, wherein the light therapy device comprises:
exactly 50% of the total LED chips having a peak emission spectra of about 630 nm;
exactly 25% of the total LED chips having a peak emission spectra of about 810 nm; and
exactly 25% of the total LED chips having a peak emission spectra of about 850 nm.

23. The method of claim 13, wherein the light therapy device comprises:
exactly 25% of the total LED chips having a peak emission spectra of about 630 nm;
exactly 25% of the total LED chips having a peak emission spectra of about 660 nm;
exactly 50% of the total LED chips having a peak emission spectra of about 850 nm.

24. The method of claim 13, wherein the light therapy device comprises:
exactly 25% of the total LED chips having a peak emission spectra of about 630 nm;
exactly 25% of the total LED chips having a peak emission spectra of about 660 nm;
exactly 50% of the total LED chips having a peak emission spectra of about 810 nm.
